(19) United States
(12) Patent Application Publication
(10) Pub. No.: US 2024/0076711 A1
(43) Pub. Date: Mar. 7, 2024

(54) IDENTIFICATION OF MITOCHONDRIA-ENRICHED CELLS

(71) Applicant: Minovia Therapeutics Ltd., Tirat Hacarmel (IL)

(72) Inventors: Natalie Yivgi-Ohana, Haifa (IL); Noa Sher, Haifa (IL)

(21) Appl. No.: 18/271,790

(22) PCT Filed: Jan. 24, 2022
(86) PCT No.: PCT/IL2022/050098

§ 371 Date: Jul. 11, 2023

Related U.S. Application Data
(60) Provisional application No. 63/141,361, filed on Jan. 25, 2021.

(51) Int. Cl. C12Q 1/32; C12N 5/073

(52) U.S. Cl. CPC C12Q 1/32; C12N 5/0605; C12Y 101/01008; C12Y 104/03004; G01N 2333/90638

(57) ABSTRACT

The present disclosure is based on the discovery that cells enriched with mitochondria are useful for treating diseases and disorders. The present invention provides methods of identifying or detecting such cells enriched with exogenous mitochondria. Specifically, the identification or detection of mitochondria-enriched cells is determined by utilization of a substrate such as tryptamine. This includes determining levels of MonoAmine oxidase A (MAO-A), Mono Amine oxidase-B (MAO-B), glycerol-3-phosphate dehydrogenase or a combination thereof. The present invention also provides kits for the identification or detection of mitochondria-enriched cells.

### BACKGROUND OF THE INVENTION

### Cross Reference to Related Applications

This application claims the benefit of U.S. Provisional Application No. 63/141,361 filed Jan. 25, 2021 under 35 U.S.C. § 119(e). The disclosure of the prior application is considered part of and is incorporated by reference in its entirety in the disclosure of this application.

### FIELD OF THE INVENTION

The present invention relates generally to cells enriched with mitochondria and more specifically methods of identifying mitochondria-enriched cells.

### BACKGROUND INFORMATION

The primary function of mitochondria is the generation of energy as adenosine triphosphate (ATP) by means of the electron-transport chain and the oxidative-phosphorylation system (the “respiratory chain”). In addition, mitochondria perform numerous essential tasks in the eukaryotic cell such as pyruvate oxidation, the Krebs cycle and metabolism of amino acids, fatty acids and steroids. Additional processes in which mitochondria are involved include heat production, storage of calcium ions, calcium signaling, programmed cell death (apoptosis) and cellular proliferation.

The ATP concentration inside the cell is typically 1-10 mM. ATP can be produced by redox reactions using simple and complex sugars (carbohydrates) or lipids as an energy source. For complex fuels to be synthesized into ATP, they first need to be broken down into smaller, simpler molecules. Complex carbohydrates are hydrolyzed into simple sugars, such as glucose and fructose. Fats (triglycerides) are metabolized to give fatty acids and glycerol.

The overall process of oxidizing glucose to carbon dioxide is known as cellular respiration and can produce about 30 molecules of ATP from a single molecule of glucose. ATP can be produced by a number of distinct cellular processes. The three main pathways used to generate energy in eukaryotic organisms are glycolysis and the citric acid cycle/oxidative phosphorylation, both components of cellular respiration, and beta-oxidation. The majority of this ATP production by non-photosynthetic eukaryotes takes place in the mitochondria, which can make up nearly 25% of the total volume of a typical cell.

Attempts to induce transfer of mitochondria into host cells or tissues have been reported. Most methods require active transfer of the mitochondria by injection. Transfer of mitochondria engulfed within a vehicle, such as a liposome, is also known. It has been shown that mtDNA transfer may occur spontaneously between cells in vitro. Further, mitochondrial transfer has been demonstrated in vitro by endocytosis or internalization.

It has been shown that mitochondria-enriched cells are useful for treating disease and disorders, specifically mitochondrial related disorders. Therefore, there is a need for methods of identifying or detecting cells enriched with exogenous mitochondria.

### SUMMARY OF THE INVENTION

The present invention provides methods of identifying or detecting cells enriched with exogenous mitochondria. The present invention also provides kits for the identification or detection of mitochondria-enriched cells.

In one embodiment, the present invention provides a method of determining enrichment of a cell with exogenous mitochondria by contacting the cell with a metabolic substrate and determining electron transfer in the cell following contacting with the metabolic substrate. In one aspect, the cells are enriched with placental mitochondria or mitochondria derived from blood. In certain aspects, the cells are stem cells, progenitor cells or bone marrow derived stem cells, pluripotent stem cells, embryonic stem cells, induced pluripotent stem cells, mesenchymal stem cells, hematopoietic stem cells, hematopoietic progenitor cells, myeloid progenitor cells, lymphoid progenitor cells, megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, myelopoietic cells, erythropoietic cells or any combination thereof. In various aspects, the cells are CD34+ cells. In one aspect, the metabolic substrate is tryptamine, D,L-a-glycerol PO₄, succinate, or a combination thereof. In an additional aspect, enzymes that utilize tryptamine are located in the mitochondria or bound to the mitochondria membrane. In a further aspect, the cells are enriched by contacting the cells with exogenous mitochondria. In one aspect, the colorimetric assay is measured by absorbance and an increased absorbance indicates the cell is enriched. In an additional aspect, contacting the cell with the metabolic substrate produces NADH and/or FADH₂.

In an additional embodiment, the present invention provides method of determining enrichment of a cell with placental mitochondria by determining levels of MonoAmine oxidase A (MAO-A) and/or MonoAmine oxidase B (MAO-B) in the cell, wherein cells enriched with placental mitochondria have increased levels of MAO-A and/or MAO-B compared with cells that are not enriched. In one aspect, the cells are stem cells, progenitor cells or bone marrow derived stem cells, pluripotent stem cells, embryonic stem cells, induced pluripotent stem cells, mesenchymal stem cells, hematopoietic stem cells, hematopoietic progenitor cells, myeloid progenitor cells, lymphoid progenitor cells, megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, myelopoietic cells, erythropoietic cells or any combination thereof. In certain aspects, the cells are CD34+ cells. In an additional aspect, the cells are enriched by contacting the cells with mitochondria. In a further aspect, the MAO-A and/or MAO-B levels are determined by mass spectroscopy.

In a further embodiment, the present invention provides a method for determining enrichment of a cell with exogenous mitochondria by determining levels of mitochondrial glycerol-3-phosphate dehydrogenase wherein cells enriched with mitochondria have increased levels of mitochondrial glycerol-3-phosphate dehydrogenase compared with cells that are not enriched. In one aspect, the cells are stem cells, progenitor cells or bone marrow derived stem cells, pluripotent stem cells, embryonic stem cells, induced pluripotent stem cells, mesenchymal stem cells, hematopoietic stem cells, hematopoietic progenitor cells, myeloid progenitor cells, lymphoid progenitor cells, megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, myelopoietic cells, erythropoietic cells or any combination thereof. In various aspects, the cells are CD34+ cells. In an additional aspect, the cells are enriched by contacting the cells with mitochondria. In various aspects, the cells are enriched with placental mitochondria or mitochondria derived from blood.

In one embodiment, the present invention provides a kit for identifying cells enriched with exogenous mitochondria with a metabolic substrate and instructions for use. In one aspect, the substrate is tryptamine, D,L-a-glycerol PO₄ or a combination thereof. In an additional aspect, mitochondria are placental mitochondria or mitochondria derived from blood.

In one embodiment, the present invention provides a method of determining enrichment of a cell with exogenous mitochondria by determining mitochondrial enrichment after contacting the cell with a metabolic substrate by colorimetric assay, determining levels of MonoAmine oxidase A (MAO-A) and/or MonoAmine oxidase B (MAO-B) in the cell, and/or determining levels of glycerol-3-phosphate dehydrogenase in the cell, wherein cells enriched with mitochondria have increased MonoAmine oxidase A (MAO-A), MonoAmine oxidase B (MAO-B), and/or glycerol-3-phosphate dehydrogenase levels, respectively, as compared with cells that are not enriched with mitochondria wherein the colorimetric assay is measured by absorbance and wherein an increase in absorbance indicates mitochondrial enrichment.

### BRIEF DESCRIPTION OF THE DRAWINGS

FIG. 1 is a schematic of the tryptamine oxidation reaction.

FIG. 2 is a schematic reaction for the formation of indole-3-acetaldehyde.

FIGS. 3A-3F show substrate utilization by isolated mitochondria. The Y-axis is the Delta OD calculated by subtracting the background values from the absorbance values. FIG. 3A: Citric acid. FIG. 3B: D,L-isocitric acid. FIG. 3C: cis-Aconitic acid. FIG. 3D: Succinic acid. FIG. 3E: Tryptamine. FIG. 3F: D,L-a-glycerol-PO₄.

FIGS. 4A-4E show substrate utilization by mitochondria-enriched cells. The Y-axis is the Delta OD calculated by subtracting the background values from the absorbance values. FIG. 4A: Tryptamine. FIG. 4B: D,L-a-glycerol-PO₄. FIG. 4C: Citric Acid. FIG. 4D: D, L-isocitric acid.

FIG. 4E: cis-Aconitic acid.

FIG. 5 shows Tryptamine utilization. The Y-axis is the Delta OD calculated by subtracting the background values from the absorbance values.

FIGS. 6A-6C are graphs showing oxygen consumption rate (OCR) of KG1a, LCL and CD34+ cells augmented with placental mitochondria. FIG. 6A: are graphs showing the OCR of KG1a cells augmented with placental mitochondria measured using substrates of complex I and complex II. FIG. 6B: are graphs showing the OCR of LCL cells augmented with placental mitochondria measured using substrates of complex I and complex II. FIG. 6C: are graphs showing the OCR of CD34+ cells augmented with placental mitochondria measured using substrates of complex I.

FIG. 7 shows tryptamine utilization by isolated mitochondria.

FIGS. 8A-B show succinate utilization. FIG. 8A: shows placenta-derived mitochondria succinate-utilization activity and blood-derived mitochondria succinate-utilization activity as assayed by MitoPlate (Biolog). FIG. 8B: shows succinate utilization activity in rising amounts of placenta-derived mitochondria particles (750k to 35M) added to the background of 50M blood-derived mitochondria particles.

### DETAILED DESCRIPTION OF THE INVENTION

The present invention provides methods of identifying or detecting cells enriched with exogenous mitochondria. Specifically, the identification or detection of mitochondria-enriched cells by determining utilization of a substrate. According to some embodiments, the identification or detection of mitochondria-enriched cells is by determining the level of an enzyme. According to some embodiments, the substrate is tryptamine, D,L-a-glycerol PO₄, succinate, or a combination thereof. According to some embodiments, levels of MonoAmine oxidase A (MAO-A), MonoAmine oxidase-B (MAO-B), or glycerol-3-phosphate dehydrogenase are determined. The present invention also provides kits for the identification or detection of mitochondria-enriched cells.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

The present invention is based in part on the finding that stem cells and bone marrow cells are receptive to being enriched with intact exogenous mitochondria and that human bone marrow cells are particularly receptive to being enriched with mitochondria as disclosed for example in WO 2016/135723. Without being bound to any theory or mechanism, it is postulated that co-incubation of cells with exogenous mitochondria promotes the transition of mitochondria into the cells. Specifically, co-incubation of stem cells or bone marrow cells with exogenous mitochondria promotes the transition of mitochondria into the stem cells or bone marrow cells.

The present invention provides methods and kits for the identification or detection of mitochondria-enriched cells.

Mitochondria play a primary role in energy production of cells. It is clear that these organelles are dynamic as the quantity and structure of the mitochondria in cells can change. Mitochondria are complex, consisting of over 1,000 proteins, the vast majority of which are encoded by nuclear rather than mitochondrial DNA. In addition to proteins, mitochondria also have specialized membranes and they can interact with each other and with other cellular organelles such as endoplasmic reticulum.

Since increasing mitochondria content in the cell results in an increase in the level of the citrate synthase (CS) activity or CoxI quantity, current solutions for verifying mitochondrial augmentation are based on quantifying the relative increase in the level of these parameters.

One of the disadvantages of using these methods is that they cannot be used independently from other methods in order to determine whether the increased mitochondrial expression in the cells is due to the exogenous mitochondria entering the cells or other reasons such as an increase in endogenous mitochondria expression (e.g. due to the stress the cell is under during augmentation).

Further, the currently available methods are based on a relative change in expression and therefore require having untreated cells as control to determine if there was an increase in the levels of CS activity or CoxI quantity in the treated cells.

The present invention demonstrates that assays to detect tryptamine utilization can be used to identify cells enriched with mitochondria. In certain aspects, the presence of MAO and/or levels of glycerol-3-phosphate dehydrogenase are determined Measuring components of these reactions provides a novel way of determining whether cells are enriched with exogenous mitochondria.

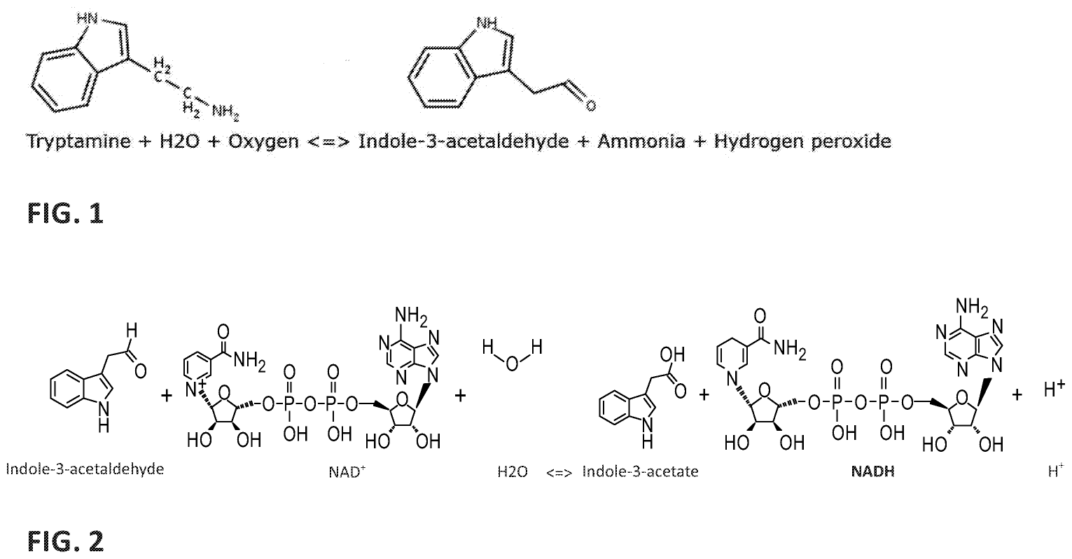

Tryptamine is a monoamine alkaloid having an indole ring structure. There are several reactions involving tryptamine Tryptamine biosynthesis generally begins from the precursor amino acid tryptophan. Tryptamine oxidation is shown in FIG. 1. This reaction can be catalyzed by two different enzymes: MonoAmine Oxidase (MAO) or Amiloride-sensitive amine oxidase (AOC1). There are two forms of MAO: MAO-A and MAO-B. MAO-A is an enzyme which catalyzes the oxidative deamination of amines, such as dopamine, norepinephrine, and serotonin. MAO-A is mainly located in the outer membrane of mitochondria but is also found in the cytosol. MAO-B catalyzes the oxidative deamination of biogenic and xenobiotic amines and plays an important role in the catabolism of neuroactive and vasoactive amines in the central nervous system and peripheral tissues (such as dopamine) and preferentially degrades benzylamine and phenethylamine. MAO-B is located in the outer membrane of mitochondria. Both MAO-A and MAO-B are also located in various tissue with high levels in the placenta. AOC1 catalyzes the degradation of compounds such as putrescine, histamine, spermine, and spermidine, substances involved in allergic and immune responses, cell proliferation, tissue differentiation, tumor formation, and possibly apoptosis. AOC1 is located in peroxisome, plasma membrane, extracellular region or secreted. AOC1 is located in various tissues with relatively medium expression levels in the placenta.

In another reaction, Indole-3-acetaldehyde formed as part of the tryptamine oxidation reaction (Reaction I) is further catalyzed by the aldehyde dehydrogenase (NAD+) family of enzymes (including ALDH2 (mitochondrial enzyme), ALDH1B1 (mitochondrial enzyme), ALDH9A1, ALDH3A2, ALDH7A1) to form NADH (FIG. 2). Some of the NAD+ family of enzymes are mitochondrial matrix enzymes, and some are cytoplasmic.

Other reactions involving tryptamine are methylation and acetylation. Methylation is catalyzed by indolethylamine N-methyltrnasferase (INMT). Acetylation is catalyzed by aralkylamine N-acetyltransferase (AANAT). These reactions and the direct downstream reactions involving the products of these reactions, do no produce NAD+ or FAD+.

Mitochondrial Glycerol-3-phosphate dehydrogenase is an enzyme that catalyzes the conversion of glycerol 3-phosphate (a.k.a D,L-glycerol-PO₄) to dihydroxyacetone phosphate (a.k.a. glycerone phosphate, outdated) coupled with reduction of FAD+ to form FADH₂.

In one embodiment, the present invention provides a method of determining enrichment of a cell with exogenous mitochondria by contacting the cell with a metabolic substrate and determining electron transfer in the cell following contacting with the metabolic substrate. In certain aspects, determining the electron transfer is by colorimetric assay, fluorescent assay, luminescent assay, or oxygen consumption. In one aspect, the cells are enriched with placental mitochondria or mitochondria derived from blood. In an additional aspect, the cells are enriched with placental mitochondria. In certain aspects, the cells are stem cells, progenitor cells or bone marrow derived stem cells, pluripotent stem cells, embryonic stem cells, induced pluripotent stem cells, mesenchymal stem cells, hematopoietic stem cells, hematopoietic progenitor cells, myeloid progenitor cells, lymphoid progenitor cells, megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, myelopoietic cells, erythropoietic cells or any combination thereof. In various aspects, the cells are CD34+ cells. In one aspect, the metabolic substrate is tryptamine, D,L-a-glycerol PO₄, succinate, or a combination thereof. In certain aspects, the metabolic substrate is tryptamine. In certain aspects, the metabolic substrate is succinate. In an additional aspect, enzymes that utilize tryptamine are located in the mitochondria or bound to the mitochondria membrane. In a further aspect, the cells were enriched by contacting the cells with exogenous mitochondria. In one aspect, the colorimetric assay is measured by absorbance and an increased absorbance indicates the cell is enriched. In an additional aspect, the contacting cells with the metabolic substrate produces NADH and/or FADH₂.

In one aspect, the isolated target cell is selected from stem cells, progenitor cells or bone marrow derived stem cells, pluripotent stem cells, embryonic stem cells, induced pluripotent stem cells, mesenchymal stem cells, hematopoietic stem cells, hematopoietic progenitor cells, myeloid progenitor cells, lymphoid progenitor cells, megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, myelopoietic cells, erythropoietic cells or any combination thereof. In a further aspect, the isolated cells are CD34+.

As used herein, the terms “enriching” or “augmenting” are used interchangeably and refer to any action designed to increase the mitochondrial content, e.g. the number of intact mitochondria, or the functionality of mitochondria of a mammalian cell. In a particular aspect, target cells enriched with exogenous mitochondria will show enhanced function compared to the same target cells prior to enrichment.

As used herein the term “target cell” is a cell that has been or will be enriched with exogenous mitochondria. In various aspects, the target cell is a stem cell, progenitor cell or bone marrow derived stem cell. Specifically, a target cell includes pluripotent stem cells, embryonic stem cells, induced pluripotent stem cells, mesenchymal stem cells, hematopoietic stem cells, hematopoietic progenitor cells, common myeloid progenitor cells, common lymphoid progenitor cells, CD34+ cells and any combination thereof.

As used herein, the term “stem cells” generally refers to any mammalian stem cells. Stem cells are undifferentiated cells that can differentiate into other types of cells and can divide to produce more of the same type of stem cells. Stem cells can be either totipotent or pluripotent.

As used herein, the term “human stem cells” generally refers to all stem cells naturally found in humans, and to all stem cells produced or derived ex vivo and are compatible with humans. In some aspects, the human stem cells are autologous. In some aspects, the human stem cells are allogeneic. A “progenitor cell”, like a stem cell, has a tendency to differentiate into a specific type of cell, but is already more specific than a stem cell and is pushed to differentiate into its “target” cell. The most important difference between stem cells and progenitor cells is that stem cells can replicate indefinitely, whereas progenitor cells can divide only a limited number of times. The term “human stem cells” as used herein further includes “progenitor cells” and “non-fully differentiated stem cells”.

In certain aspects, the stem cells are pluripotent stem cells (PSC). In some aspects, the stem cells are induced PSCs (iPSCs). In certain aspects, the stem cells are embryonic stem cells. In certain aspects, the stem cells are derived from bone-marrow cells. In particular aspects, the stem cells are CD34+ cells. In particular aspects, the stem cells are mesenchymal stem cells. In other aspects, the stem cells are derived from adipose tissue. In yet other aspects, the stem cells are derived from blood. In further aspects, the stem cells are derived from umbilical cord blood. In further aspects, the stem cells are derived from oral mucosa. In specific aspects, the stem cells obtained from a patient afflicted with a disease of disorder or from a healthy subject are bone marrow cells or bone marrow-derived stem cells.

As used herein the term “pluripotent stem cells (PSCs)” refers to cells that can propagate indefinitely, as well as give rise to a plurality of cell types in the body. Totipotent stem cells are cells that can give rise to every other cell type in the body. Embryonic stem cells (ESCs) are totipotent stem cells and induced pluripotent stem cells (iPSCs) are pluripotent stem cells.

As used herein the term “induced pluripotent stem cells (iPSCs)” refers to a type of pluripotent stem cell that can be generated from human adult somatic cells. Some non-limiting examples of somatic cells from which iPSC can be generated herein include fibroblast cells, endothelial cells, capillary blood cells, keratinocytes, myeloid cells epithelial cells.

As used herein the term “embryonic stem cells (ESC)” refers to a type of totipotent stem cell derived from the inner cell mass of a blastocyst.

As used herein the term “bone marrow cells” generally refers to all human cells naturally found in the bone marrow of humans, and to all cell populations naturally found in the bone marrow of humans. The term “bone marrow stem cells” and “bone marrow-derived stem cells” refer to the stem cell population derived from the bone marrow.

In some aspects, the target cells are pluripotent stem cells, embryonic stem cells, induced pluripotent stem cells, mesenchymal stem cells, hematopoietic stem cells, hematopoietic progenitor cells, common myeloid progenitor cells, common lymphoid progenitor cells, CD34+ cells and any combination thereof.

In some aspects, the autologous or allogeneic human stem cells are pluripotent stem cells (PSCs) or induced pluripotent stem cells (iPSCs). In further aspects, the autologous or allogeneic human stem cells are mesenchymal stem cells.

According to several aspects, the human stem cells are derived from adipose tissue, oral mucosa, blood, umbilical cord blood or bone marrow. In specific aspects, the human stem cells are derived from bone marrow.

In certain aspects, the bone-marrow derived stem cells include myelopoietic cells. The term “myelopoietic cells” as used herein refers to cells involved in myelopoiesis, e.g. in the production of bone-marrow and of all cells that arise from it, namely, all blood cells.

In certain aspects, the bone-marrow derived stem cells include erythropoietic cells. The term “erythropoietic cells” as used herein refers to cells involved in erythropoiesis, e.g. in the production of red blood cells (erythrocytes).

In certain aspects, the bone-marrow derived stem cells include multi-potential hematopoietic stem cells (HSCs). The term “multi-potential hematopoietic stem cells” or “hemocytoblasts” as used herein refers to the stem cells that give rise to all the other blood cells through the process of hematopoiesis.

In certain aspects, the bone-marrow derived stem cells comprise common myeloid progenitor cells, common lymphoid progenitor cells, or any combination thereof. In certain aspects, the bone-marrow derived stem cells comprise mesenchymal stem cells. The term “common myeloid progenitor” as used herein refers to the cells that generate myeloid cells. The term “common lymphoid progenitor” as used herein refers to the cells that generate lymphocytes.

In certain aspects, the bone-marrow derived stem cells further comprise megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, or any combination thereof.

In certain aspects, the bone-marrow derived stem cells include mesenchymal stem cells. The term “mesenchymal stem cells” as used herein refers to multipotent stromal cells that can differentiate into a variety of cell types, including osteoblasts, chondrocytes, myocytes and adipocytes.

In certain aspects, the bone-marrow derived stem cells include myelopoietic cells. In certain aspects, the bone-marrow derived stem cells consist of erythropoietic cells. In certain aspects, the bone-marrow derived stem cells include multi-potential hematopoietic stem cells (HSCs). In certain aspects, the bone-marrow derived stem cells include common myeloid progenitor cells, common lymphoid progenitor cells, or any combination thereof. In certain aspects, the bone-marrow derived stem cells include megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, or any combination thereof. In certain aspects, the bone-marrow derived stem cells consist of mesenchymal stem cells. In certain aspects, the stem cells include a plurality of human bone marrow stem cells obtained from peripheral blood.

Hematopoietic progenitor cell antigen CD34, also known as CD34 antigen, is a protein that in humans is encoded by the CD34 gene. CD34 is a cluster of differentiation in a cell surface glycoprotein and functions as a cell-cell adhesion factor. In certain aspects, the bone-marrow stem cells express the bone-marrow progenitor cell antigen CD34 (are CD34+). In certain aspects, the bone marrow stem cells present the bone-marrow progenitor cell antigen CD34 on their external membrane. In certain aspects, the CD34+ cells are from umbilical cord blood.

As used herein the term “CD34+ cells” refers to hematopoietic stem cells characterized as being CD34 positive, regardless of their origin. In certain aspects, the CD34+ cells are obtained from the bone marrow, from bone marrow cells mobilized to the blood, or obtained from umbilical cord blood.

As used herein the phrase “stem cells obtained from a subject afflicted with a disorder or from a donor not afflicted with a disorder” refers to cells that were stem cells in the subject/donor at the time of their isolation from the subject.

As used herein the phrase “stem cells derived from a subject afflicted with a disorder” or “from a donor not afflicted with a disorder” refers to cells that were not stem cells in the subject/donor, and have been manipulated to become stem cells. The term “manipulated” as used herein refers to the use of any one of the methods known in the field (Yu J. et al., Science, 2007, Vol. 318(5858), pages 1917-1920) for reprograming somatic cells to an undifferentiated state and becoming induced pluripotent stem cells (iPSCs), and, optionally, further reprograming the iPSCs to become cells of a desired lineage or population (Chen M. et al., IOVS, 2010, Vol. 51(11), pages 5970-5978), such as bone marrow cells (Xu Y. et al., PLoS ONE, 2012, Vol. 7(4), page e34321).

In certain aspects, the stem cells are directly derived from the subject afflicted with a disease or disorder. In certain aspects, the stem cells are directly derived from a donor. The term “directly derived” as used herein refers to stem cells which were derived directly from other cells. In certain aspects, the hematopoietic stem cells (HSC) were derived from bone-marrow cells. In certain aspects, the hematopoietic stem cells (HSC) were derived from peripheral blood.

In certain aspects, the stem cells are indirectly derived from the subject afflicted with a disease or disorder. In certain aspects, the stem cells are indirectly derived from a donor. The term “indirectly derived” as used herein refers to stem cells which were derived from non-stem cells. In certain aspects, the stem cells were derived from somatic cells which were manipulated to become induced pluripotent stem cells (iPSCs).

In some aspects, the target cells are obtained from whole blood, blood fractions, peripheral blood, PBMC, serum, plasma, adipose tissue, oral mucosa, blood, umbilical cord blood or bone marrow. In certain aspects, the stem cells are directly obtained from the bone marrow of the subject afflicted with a disease or disorder. In certain aspects, the stem cells are directly obtained from the bone-marrow of a donor. The term “directly obtained” as used herein refers to stem cells which were obtained from the bone-marrow itself, e.g. by means such as surgery or suction through a needle by a syringe.

In certain aspects, the target cells are indirectly obtained from the bone marrow of the patient afflicted with a disease or disorder. In certain aspects, the target cells are indirectly obtained from the bone marrow of a donor. The term “indirectly obtained” as used herein refers to bone marrow cells which were obtained from a location other than the bone marrow itself.

In certain aspects, the target cells are obtained from the peripheral blood of the subject afflicted with a disease or disorder. In certain aspects, the target cells are obtained from the peripheral blood of a healthy donor or subject. The term “peripheral blood” as used herein refers to blood circulating in the blood system.

As used herein, the terms “autologous cells” or “cells that are autologous” are used interchangeably and refer to the patient's own cells.

In an additional aspect, the isolated target cells are genetically modified cells. In certain aspects, the genetically modified cells are T-cells. In certain aspects, the genetically modified cells are T-cell receptor (TCR) or chimeric antigen receptor (CAR)-transduced T cells.

The term “lymphocytes” as used herein refers to white blood cells that play a major role in defending the body against disease, and includes T-cells, natural killer cells (NK cells), B-cells and mixtures thereof. The above listed immune cell types can be divided into further subsets. In some aspects, the lymphocytes are mature lymphocytes. In some aspects, the lymphocytes are non-genetically modified lymphocytes. In other aspects, the lymphocytes are genetically modified lymphocytes.

The terms “T cells” and “T lymphocytes” are used interchangeably herein. T cell is specific type of lymphocyte that has an important role in controlling and shaping the immune response by providing a variety of immune-related functions. T cells can be distinguished from other lymphocytes by the presence of a T-cell receptor (TCR) on the cell surface. The term “T cells” as used herein includes cytotoxic T cells, T helper cells, regulatory T cells and natural killer T cells (NKT). According to some aspects, the T cells are T cells precursors. According to other aspects, the T cells are mature T cells. According to some aspects, the T cells are fully differentiated T cells.

According to some aspects, the target cells are derived from a mammalian subject, preferably a human subject.

As used herein, the terms “mitochondria-enriched cell” or “mitochondria-enriched target cell” are used interchangeably and refer to any cell that has exogenous mitochondria inserted. The cell may be a target cell. The cell may be a stem cell.

As used herein the term, “mitochondria-enriched T cells” refers T cells with exogenous mitochondria inserted.

As used herein the term, “mitochondria-enriched hematopoietic stem cells” is a hematopoietic stem cell with exogenous mitochondria inserted.

The term “allogeneic cells” refers to cells being from a source other than the subject such as a different donor individual.

The term “syngeneic” as used herein and in the claims refers to genetic identity or genetic near-identity sufficient to allow grafting among individuals without rejection. The term syngeneic in the context of mitochondria is used herein interchangeably with the term autologous mitochondria meaning of the same maternal bloodline.

In certain aspects, the mitochondria-enriched target cells, which may be stem cells, have at least one of (i) an increased mitochondrial DNA content compared to the mitochondrial DNA content in the target cells prior to mitochondrial enrichment; (ii) an increased rate of oxygen (O₂) consumption compared to the rate of oxygen (O₂) consumption in target cells prior to mitochondrial enrichment; (iii) an increased content or activity level of citrate synthase compared to the content or activity level of citrate synthase in target cells prior to mitochondrial enrichment; (iv) an increased rate of adenosine triphosphate (ATP) production compared to the rate of adenosine triphosphate (ATP) production in target cells prior to mitochondrial enrichment; (v) a lower level of heteroplasmy; or any combination of (i), (ii), (iii) (iv) and (v).

In certain aspects, the target cells are allogeneic to the subject afflicted with the disorder. The term “allogeneic to the subject” refers to the stem cells or mitochondria being HLA matched to the cells of the patient or at least partially HLA matched. According to certain aspects, the donor is matched to the subject according to identification of a specific mitochondrial DNA haplogroup. In certain aspects, the subject is the source of stem cells and/or mitochondria.

The term “HLA-matched” as used herein refers to the desire that the subject and the donor of the target cells be as closely HLA-matched as possible, at least to the degree in which the subject does not develop an acute immune response against the target cells of the donor. The prevention and/or therapy of such an immune response may be achieved with or without acute or chronic use of immune-suppressors. In certain aspects, target cells from a donor are HLA-matched to the patient to a degree wherein the patient does not reject the target cells.

The term “haplogroup” as used herein refers to a genetic population group of people who share a common ancestor on the matriline. Mitochondrial haplogroup is determined by sequencing.

In certain aspects, the mitochondria are from identical haplogroups. In other aspects, the mitochondria are from different haplogroups.

In some aspects, the target cells are cultured and expanded in vitro. In certain aspects, the target cells undergo at least one freeze thaw cycle prior to or following mitochondrial enrichment.

In various aspects, the exogenous mitochondria are isolated from the subject or from a donor. According to certain aspects, the exogenous mitochondria are isolated from a donor selected from a specific mitochondria haplogroup, in accordance with the disorder of the subject.

In various aspects, the exogenous mitochondria are fresh, frozen or freeze-thawed.

As used herein the term “donor” refers to a donor providing the exogenous mitochondria. In some aspects, the donor is not suffering from a disease or disorder or is not suffering from the same disease or disorder which the subject is afflicted.

The term “exogenous” or “isolated exogenous” with regard to mitochondria refers to mitochondria that are introduced to a target cell (for example, stem cells), from a source which is external to the cell. For example, in some aspects, exogenous mitochondria are commonly derived or isolated from a donor cell which is different than the target cell. For example, exogenous mitochondria may be produced or made in a donor cell, purified, isolated or obtained from the donor cell and thereafter introduced into the target cell. Exogenous mitochondria can be allogenic as obtained from a donor or autologous as obtained from a subject. Isolated mitochondria may include functional mitochondria. In certain aspects, the exogenous mitochondria are whole mitochondria.

As used herein, the terms “isolated” and “partially purified” in the context of mitochondria includes exogenous mitochondria that were purified, at least partially, from other cellular components. The total amount of mitochondrial proteins in an exogenous isolated or partially purified mitochondria is between 10%-90% of the total amount of cellular proteins within the sample.

As used herein the term “functional mitochondria” refers to mitochondria displaying parameters indicative of normal mitochondrial DNA (mtDNA) and normal, non-pathological levels of activity. The activity of mitochondria can be measured by a variety of methods well known in the art, such as membrane potential, O₂ consumption, ATP production, and citrate synthase (CS) activity level.

In certain aspects, the exogenous mitochondria constitute at least 1% of the total mitochondria content in the mitochondria-enriched cell. In certain aspects, the exogenous mitochondria constitute at least 10% of the total mitochondria content in the mitochondria-enriched target cell. In some aspects, the exogenous mitochondria constitute at least about 3%, 5%, 10%, 15%, 20%, 25%, 30%, 40% or 50% of the total mitochondria content in the mitochondria-enriched target cell. In certain aspects, the total amount of mitochondrial proteins in the isolated mitochondria, is between 10-90%, 20-80%, 20-70%, 40-70%, 20-40%, or 20-30% of the total amount of cellular proteins. In certain aspects, the total amount of mitochondrial proteins in the isolated mitochondria, is between 20%-80% of the total amount of cellular proteins within the sample. In certain aspects, the total amount of mitochondrial proteins in the isolated mitochondria, is between 20%-80% of the combined weight of the mitochondria and other sub-cellular fractions. In other aspects, the total amount of mitochondrial proteins in the isolated mitochondria, is above 80% of the combined weight of the mitochondria and other sub-cellular fractions.

In certain aspects, the exogenous mitochondria are obtained from a human cell or a human tissue. In some aspects, the human cell or human tissue is selected from placental cells, placental cells grown in culture, and blood cells. In some aspects, the human cell is a human stem cell. In some embodiments, the human cell is a human somatic cell. In some aspects, the cells are cells in culture. Some non-limiting examples of somatic cells include fibroblast cells, endothelial cells, capillary blood cells, keratinocytes, myeloid cells, and epithelial cells.

The term “autologous” with regards to mitochondria refers to mitochondria that are introduced to a target cell (for example, stem cells), from a source which is the same as the cell. For example, in some aspects, autologous mitochondria are derived or isolated from a subject that is the source of the target cell. For example, autologous mitochondria may be purified/isolated/obtained from the subject's cell and thereafter introduced into the target cell of the subject. The term “autologous mitochondria”, refers to mitochondria obtained from the patient's own cells or from maternally related cells. The term “allogeneic mitochondria” refers to mitochondria being from a different donor individual.

The term “endogenous” with regard to mitochondria refers to mitochondria that is being made/expressed/produced by a cell and is not introduced from an external source into the cell. In some aspects, endogenous mitochondria contain proteins and/or other molecules which are encoded by the genome of the cell. In some aspects, the term “endogenous mitochondria” is equivalent to the term “host mitochondria”.

In certain aspects, exogenous human mitochondria are introduced into target cells which may be human stem cells, thus enriching these cells with exogenous mitochondria. In certain aspects, the target cells are enriched with the exogenous mitochondria by contacting or incubating the target cells with the exogenous mitochondria. The contacting or incubating is performed under conditions allowing the exogenous or isolated mitochondria to enter the target cells.

It should be understood that such enrichment changes the mitochondrial content of the target cells: while naive human target cells substantially have one population of host/autologous mitochondria, target cells enriched with exogenous mitochondria substantially have two populations of mitochondria, a first population of host/endogenous mitochondria and another population of the introduced mitochondria (i.e., the exogenous mitochondria). Thus, the term “enriched” relates to the state of the cells after receiving/incorporation exogenous mitochondria. Determining the number and/or ratio between the two populations of mitochondria is straightforward, as the two populations may differ in several aspects e.g. in their mitochondrial DNA. Therefore, the phrase “human cells enriched with exogenous human mitochondria” is equivalent to the phrase “human cells comprising endogenous mitochondria and exogenous isolated mitochondria”. For example, human target cells which comprise at least 1% exogenous isolated mitochondria of the total mitochondria content, are considered comprising host endogenous mitochondria and exogenous isolated mitochondria in a ratio of 99:1. For example, “3% of the total mitochondria” means that after enrichment the original (endogenous) mitochondrial content is 97% of the total mitochondria and the introduced (exogenous) mitochondria is 3% of the total mitochondria—this is equivalent to (3/97=) 3.1% enrichment. In another example, “33% of the total mitochondria” means that after enrichment, the original (endogenous) mitochondrial content is 67% of the total mitochondria and the introduced (exogenous) mitochondria is 33% of the total mitochondria—this is equivalent to (33/67=) 49.2% enrichment.

In some aspects, the identification/discrimination of endogenous mitochondria from exogenous mitochondria, after the latter have been introduced into the target cell, can be performed by various means, including, for example, but not limited to: identifying differences in mtDNA sequences, for example different haplotypes, between the endogenous mitochondria and exogenous mitochondria, identifying specific mitochondrial proteins originating from the source tissue of the exogenous mitochondria, such as, for example, cytochrome p450 cholesterol side chain cleavage (P450SCC) from placenta, UCP1 from brown adipose tissue, and the like, or any combination thereof.

Heteroplasmy is the presence of more than one type of mitochondrial DNA within a cell or individual. The heteroplasmy level is the proportion of mutant mtDNA molecules vs. wild type/functional mtDNA molecules and is an important factor in considering the severity of mitochondrial diseases. While lower levels of heteroplasmy (sufficient amount of mitochondria are functional) are associated with a healthy phenotype, higher levels of heteroplasmy (insufficient amount of mitochondria are functional) are associated with pathologies. In certain aspects, the heteroplasmy level of the enriched stem cells is at least 1%, 3%, 5%, 15%, 20%, 25%, or 30% lower than the heteroplasmy level of the stem cells obtained or derived from the subject or donor.

As used herein the terms “mitochondria-enriched target cells” or “mitochondria-enriched cells” are used interchangeably and refer to a target cell that has had exogenous mitochondria inserted. In certain aspects, the mitochondria-enriched target cells differentiate to CD45, CD3, CD33, CD14, CD19, CD11, CD15, CD16 and the like expressing cells. In certain aspects, the mitochondria-enriched target cells express CD45, CD3, CD33, CD14, or CD19. CD45 is a receptor linked protein tyrosine phosphatase present in all cells of the hematopoietic lineage except erythrocytes and plasma cells. CD3 is a marker of immune response efficiency. Specifically, CD3 is expressed in pro-thymocytes. Expression of CD45 and CD3 on cells can be determined by any means known in the art including flow cytometry.

In some aspects, the methods described above further includes expanding the target cells by culturing said stem cells in a proliferation medium capable of expanding the target cells. In other aspects, the method further comprises expanding the mitochondria-enriched target cells by culturing said cells in a culture or proliferation medium capable of expanding target cells. As used throughout this application, the term “culture or proliferation medium” is a fluid medium such as cell culture media, cell growth media, buffer which provides sustenance to the cells.

As used herein the term “contacting” refers to bringing the mitochondria and cells into sufficient proximity to promote entry of the mitochondria into the cells. The term introducing or inserting mitochondria into the target cells is used interchangeably with the term contacting.

The phrase “conditions allowing the isolated mitochondria to enter the target cells” as used herein generally refers to parameters such as time, temperature, culture medium and proximity between the mitochondria and the stem cells. For example, human cells and human cell lines are routinely incubated in liquid medium, and kept in sterile environments, such as in tissue culture incubators, at 37° C. and 5% CO₂ atmosphere. According to alternative aspects, disclosed and exemplified herein the cells may be incubated at room temperature in saline supplemented with human serum albumin.

In certain aspects, the human target cells are incubated with the isolated mitochondria for a time ranging from 0.5 to 30 hours, at a temperature ranging from about 16 to about 37° C. In certain aspects, the human target cells are incubated with the isolated mitochondria for a time ranging from 1 to 30 or from 5 to 25 hours. In specific aspects, incubation is for 20 to 30 hours. In some aspects, incubation is for at least 1, 3, 5, 8, 10, 13, 15, 18, 20, 21, 22, 23 or 24 hours. In other aspects, incubation is up to 5, 10, 15, 20 or 30 hours. In specific aspects, incubation is for 24 hours. In certain aspects, incubation is until the mitochondrial content in the target cells is increased in average by 1% to 45% compared to their initial mitochondrial content.

In some aspects, incubation is at room temperature (16° C. to 30° C.). In other aspects, incubation is at 37° C. In some aspects, incubation is in a 5% CO₂ atmosphere. In other aspects, incubation does not include added CO₂ above the level found in air.

In yet further aspects, the incubation is performed in culture medium supplemented with human serum albumin (HSA). In additional aspects, the incubation is performed in saline supplemented with HSA. According to certain exemplary aspects, the conditions allowing the isolated exogenous mitochondria to enter the human stem cells thereby enriching said human target cells with said human exogenous mitochondria include incubation at room temperature in saline supplemented with 4.5% human serum albumin.

In one aspect, the mitochondria are obtained from a donor. In another aspect, the exogenous mitochondria are autologous or allogenic to the target cell.

In certain aspects, the incubation is performed at room temperature. In certain aspects, the incubation is performed for at least 6 hours. In certain aspects, the incubation is performed for at least 12 hours. In certain aspects, the incubation is performed for 12 to 24 hours. In certain aspects, the conditions are sufficient to increase the mitochondrial content of the naive target cells by at least about 1%, 3%, 5% or 10% as determined by CS activity.

Citrate synthase (CS) is localized in the mitochondrial matrix but is encoded by nuclear DNA. Citrate synthase is involved in the first step of the Krebs cycle, and is commonly used as a quantitative enzyme marker for the presence of intact mitochondria (Larsen S. et al., J. Physiol., 2012, Vol. 590(14), pages 3349-3360; Cook G. A. et al., Biochim. Biophys. Acta., 1983, Vol. 763(4), pages 356-367).

Mitochondrial dose can be expressed in terms of units of CS activity or mtDNA copy number of other quantifiable measurements of the amount of exogenous mitochondria as explained herein. A “unit of CS activity” is defined as the amount that enables conversion of one micromole substrate in 1 minute in 1 mL reaction volume.

In some aspects, the enrichment of the target cells with exogenous mitochondria includes introducing into the target cells a dose of mitochondria of at least 0.044 up to 176 milliunits (mU) of citrate synthase (CS) activity per million cells; at least 0.088 up to 176 mU of CS activity per million cells; at least 0.2 up to 150 mU of CS activity per million cells; at least 0.4 up to 100 mU of CS activity per million cells; at least 0.6 up to 80 mU of CS activity per million cells; at least 0.7 up to 50 mU of CS activity per million cells; at least 0.8 up to 20 mU of CS activity per million cells; at least 0.88 up to 17.6 mU of CS activity per million cells; or at least 0.44 up to 17.6 milliunits of CS activity per million cells.

As used herein the term “mitochondrial content” refers to the amount of mitochondria within a cell, or to the average amount of mitochondria within a plurality of cells. The term “increased mitochondrial content” as used herein refers to a mitochondrial content which is detectably higher than the mitochondrial content of the target cells prior to mitochondria enrichment.

In certain aspects, the mitochondrial content of the human target cells enriched with exogenous mitochondria is detectably higher than the mitochondrial content of the target cells prior to enrichment. According to various aspects, the mitochondrial content of the mitochondria-enriched target cells is at least 1%, at least 3%, at least 5%, at least 10%, at least 25%, at least 50%, at least 100%, at least 200% or more, higher than the mitochondrial content of the target cells.

In certain aspects, the mitochondrial content of the target cells or mitochondria-enriched target cells is determined by determining the content of citrate synthase. In certain aspects, the mitochondrial content of the target cells or enriched stem cells is determined by determining the activity level of citrate synthase. In certain aspects, the mitochondrial content of the target cells or enriched target cells correlates with the content of citrate synthase. In certain aspects, the mitochondrial content of the target cells or enriched target cells correlates with the activity level of citrate synthase. CS activity can be measured by commercially available kits e.g., using the CS activity kit CS0720 (Sigma).

Mitochondrial DNA content may be measured by performing quantitative PCR of a mitochondrial gene prior and post mitochondrial enrichment, normalized to a nuclear gene.

In specific situations the same cells, prior to mitochondria enrichment, serve as controls to measure additional parameters such as CS and ATP activity and determine enrichment level.

In certain aspects, the term “detectably higher” as used herein refers to a statistically-significant increase between the normal and increased values. In certain aspects, the term “detectably higher” as used herein refers to a non-pathological increase, i.e. to a level in which no pathological symptom associated with the substantially higher value becomes apparent. In certain aspects, the term “increased” as used herein refers to a value which is 1.05 fold, 1.1 fold, 1.25 fold, 1.5 fold, 2 fold, 3 fold, 4 fold, 5 fold, 6 fold, 7 fold or higher than the corresponding value found in corresponding cells or corresponding mitochondria of a healthy subject or of a plurality of healthy subjects or in the target cells prior to mitochondrial enrichment.

The term “increased mitochondrial DNA content” as used herein refers to the content of mitochondrial DNA which is detectably higher than the mitochondrial DNA content in target cells prior to mitochondria enrichment. Mitochondrial content may be determined by measuring SDHA or COX1 content. “Normal mitochondrial DNA” in the context of the specification and claims refers to mitochondrial DNA not carrying/having a mutation or deletion that is known to be associated with a mitochondrial disease. The term “normal rate of oxygen (O₂) consumption” as used herein refers to the average 02 consumption of cells from healthy individuals. The term “normal activity level of citrate synthase” as used herein refers to the average activity level of citrate synthase in cells from healthy individuals. The term “normal rate of adenosine triphosphate (ATP) production” as used herein refers to the average ATP production rate in cells from healthy individuals.

In some embodiments, the extent of enrichment of the target cells with exogenous mitochondria may be further determined by functional and/or enzymatic assays, including but not limited to rate of oxygen (O₂) consumption, content or activity level of citrate synthase, rate of adenosine triphosphate (ATP) production. In the alternative the enrichment of the target cells with exogenous mitochondria may be confirmed by the detection of mitochondrial DNA of the donor. According to some aspects, the extent of enrichment of the target cells with exogenous mitochondria may be determined by the level of change in heteroplasmy and/or by the copy number of mtDNA per cell.

TMRM (tetramethylrhodamine methyl ester) or the related TMRE (tetramethylrhodamine ethyl ester) are cell-permeant fluorogenic dyes commonly used to assess mitochondrial function in living cells, by identifying changes in mitochondrial membrane potential. According to some aspects, the level of enrichment can be determined by staining with TMRE or TMRM.

According to some aspects, the intactness of a mitochondrial membrane may be determined by any method known in the art. In a non-limiting example, intactness of a mitochondrial membrane is measured using the tetramethylrhodamine methyl ester (TMRM) or the tetramethylrhodamine ethyl ester (TMRE) fluorescent probes. Mitochondria that were observed under a microscope and show TMRM or TMRE staining have an intact mitochondrial outer membrane. As used herein, the term “a mitochondrial membrane” refers to a mitochondrial membrane selected from the group consisting of the mitochondrial inner membrane, the mitochondrial outer membrane, and both.

In certain aspects, the level of mitochondrial enrichment in the mitochondria-enriched human target cells is determined by sequencing at least a statistically-representative portion of total mitochondrial DNA in the cells and determining the relative levels of host/endogenous mitochondrial DNA and exogenous mitochondrial DNA. In certain aspects, the level of mitochondrial enrichment in the mitochondria-enriched human target cells is determined by single nucleotide polymorphism (SNP) analysis. In certain aspects, the largest mitochondrial population and/or the largest mitochondrial DNA population is the host/endogenous mitochondrial population and/or the host/endogenous mitochondrial DNA population; and/or the second-largest mitochondrial population and/or the second-largest mitochondrial DNA population is the exogenous mitochondrial population and/or the exogenous mitochondrial DNA population.

According to certain aspects, the enrichment of the target cells with exogenous mitochondria may be determined by conventional assays that are recognized in the art. In certain aspects, the level of mitochondrial enrichment in the mitochondria-enriched human target cells is determined by (i) the levels of host/endogenous mitochondrial DNA and exogenous mitochondrial DNA; (ii) the level of mitochondrial proteins selected from the group consisting of citrate synthase (CS), cytochrome C oxidase (COX1), succinate dehydrogenase complex flavoprotein subunit A (SDHA) and any combination thereof; (iii) the level of CS activity; or (iv) any combination of (i), (ii) and (iii). In certain aspects, the level of enrichment in the mitochondria-enriched human target cells is determined by determining utilization of a substrate such as tryptamine by determining levels of NADH, FADH₂, MAO-A, MAO-B, glycerol-3-phosphate dehydrogenase or any combination thereof. In certain aspects, the level of enrichment in the mitochondria-enriched human target cells is determined by determining utilization of a substrate such as succinate by determining, for example, levels of dehydrogenase complex flavoprotein subunit A.

In certain aspects, the level of mitochondrial enrichment in the mitochondria-enriched human stem cells is determined by at least one of: (i) the levels of host mitochondrial DNA and exogenous mitochondrial DNA in case of allogeneic mitochondria; (ii) the level of citrate synthase activity; (iii) the level of succinate dehydrogenase complex flavoprotein subunit A (SDHA) or cytochrome C oxidase (COX1); (iv) the rate of oxygen (O₂) consumption; (v) the rate of adenosine triphosphate (ATP) production; (vi) determining tryptamine utilization (vii) determining succinate utilization (viii) determining rates of electron flow into and through the electron transport chain (ETC) from the metabolic substrate that produce NADH or FADH₂, MAO-A, MAO-B, or glycerol-3-phosphate dehydrogenase or (ix) any combination thereof. Methods for measuring these various parameters are well known in the art. It should be understood that methods described herein can be used independently or in combination.

In some aspects, enrichment of the target cells with exogenous human mitochondria comprises washing the mitochondria-enriched target cells after incubation of the human target cells with said isolated exogenous human mitochondria. This step provides mitochondria-enriched target cells substantially devoid of cell debris or mitochondrial membrane remnants and mitochondria that did not enter the target cells. In some aspects, washing comprises centrifugation of the mitochondria-enriched target cells after incubation of the human target cells with said isolated exogenous human mitochondria. According to some aspects, mitochondria-enriched human cells are separated from free mitochondria, i.e., mitochondria that did not enter the stem cells, or other cell debris.

Clearing residuals from the composition comprising the mitochondria-enriched cells can be performed using different methods known in the art. According to some aspects, residuals clearing is done by centrifugation.

In certain aspects, the target cells and/or the isolated exogenous mitochondria are concentrated before or during incubation and/or contacting. In certain aspects, the target cells and/or isolated exogenous mitochondria are subjected to centrifugation before, during or after incubation or contacting. In some aspects, there is a single or more centrifugation steps before, during or after incubation of the target cells with the isolated mitochondria.

In certain aspects, the centrifugation speed is 7,000 g or 8,000 g. According to further aspects, the centrifugation is at a speed between 300 g-8000 g; 500 g-8000 g; 1000 g-8000 g; 300 g-5000 g; 2000 g-4000 g; 2500 g-8500 g; 3000 g-8000 g; 4000 g-8000 g; 5,000-10,000 g 7000 g-8000 g or above 2500 g. In some aspects, centrifugation is performed for a time ranging from 2 minutes to 30 minutes; 3 minutes to 25 minutes; 5 minutes to 20 minutes; or 8 minutes to 15 minutes.

In some aspects, centrifugation is performed in a temperature ranging from 2 to 6° C.; 4 to 37° C.; 4 to 10° C. or 16 to 30° C. In specific aspects, centrifugation is performed at 4° C. In some aspects, there is a centrifugation step before, during or after incubation of the target cells with the isolated exogenous mitochondria, followed by resting the cells at a temperature lower than 30° C. In some aspects, the conditions allowing the isolated exogenous mitochondria to enter the human target cells include a single centrifugation before, during or after incubation of the target cells with the isolated mitochondria, followed by resting the cells at a temperature ranging between 16 to 28° C.

In certain aspects, the target cells are used fresh. In some aspects, the target cells are frozen and thawed prior to or following enrichment with mitochondria.

In certain aspects, target cells are fresh. In certain aspects, the target cells are frozen and then thawed prior to incubation. In certain aspects, the isolated exogenous mitochondria are fresh. In certain aspects, the isolated exogenous mitochondria are frozen and then thawed prior to incubation. In certain aspects, the mitochondria-enriched target cells are fresh. In certain aspects, the mitochondria-enriched target cells are frozen. In certain aspects, the mitochondria-enriched target cells are frozen and then thawed.

In certain aspects, the mitochondria are not frozen. In further aspects, the isolated mitochondria are frozen, then stored and thawed prior to use. In further aspects, the mitochondria-enriched target cells are used without freezing and storage. In yet further aspects, the mitochondria-enriched target cells are used after freezing, storage and thawing. Methods suitable for freezing and thawing of cell preparations in order to preserve viability are well known in the art.

As used herein, the term “freeze-thaw cycle” refers to freezing of the isolated exogenous mitochondria to a temperature below 0° C., maintaining the mitochondria in a temperature below 0° C. for a defined period of time and thawing the isolated mitochondria to room temperature or body temperature or any temperature above 0° C. which enables contacting the target cells with the isolated mitochondria. The term “room temperature”, as used herein typically refers to a temperature of between 18° C. and 25° C. The term “body temperature”, as used herein, refers to a temperature of between 35.5° C. and 37.5° C., preferably 37° C.

In another aspect, the mitochondria that have undergone a freeze-thaw cycle were frozen at a temperature of −20° C. or lower; −4° C. or lower; or −70° C. or lower. According to another aspect, freezing of the mitochondria is gradual. According to some aspects, freezing of mitochondria is through flash-freezing. As used herein, the term “flash-freezing” refers to rapidly freezing the mitochondria by subjecting them to cryogenic temperatures.

In another aspect, the mitochondria that underwent a freeze-thaw cycle were frozen for at least 30 minutes prior to thawing. According to another aspect, the freeze-thaw cycle comprises freezing the isolated exogenous mitochondria for at least 30, 60, 90, 120, 180, 210 minutes prior to thawing. In another aspect, the isolated exogenous mitochondria that have undergone a freeze-thaw cycle were frozen for at least 1, 2, 3, 4, 5, 6, 7, 8, 9, 10, 24, 48, 72, 96, or 120 hours prior to thawing. In another aspects, the isolated exogenous mitochondria that have undergone a freeze-thaw cycle were frozen for at least 4, 5, 6, 7, 30, 60, 120, 365 days prior to thawing. According to another aspect, the freeze-thaw cycle comprises freezing the isolated exogenous mitochondria for at least 1, 2, 3 weeks prior to thawing. According to another aspect, the freeze-thaw cycle comprises freezing the isolated exogenous mitochondria for at least 1, 2, 3, 4, 5, 6 months prior to thawing. According to another aspect, the oxygen consumption of the isolated exogenous mitochondria after the freeze-thaw cycle is equal or higher than the oxygen consumption of the exogenous mitochondria prior to the freeze-thaw cycle.

According to certain aspects, thawing is at room temperature. In another aspect, thawing is at body temperature. According to another aspect, thawing is at a temperature which enables contacting or incubating the exogenous mitochondria with the target cells. According to another aspect, thawing is performed gradually.

As used herein, a “sample” or “biological sample” is meant to refer to any “biological specimen” collected from a subject, and that is representative of the content or composition of the source of the sample, considered in its entirety. A sample can be collected and processed directly for analysis, or be stored under proper storage conditions to maintain sample quality until analyses are completed. Ideally, a stored sample remains equivalent to a freshly-collected specimen. The source of the sample can be an internal organ, vein, artery, or even a fluid. Non-limiting examples of sample include blood, plasma, urine, saliva, sweat, organ biopsy, cerebrospinal fluid (CSF), tear, semen, vaginal fluid, feces, skin, and hair.

In certain aspects, the subject afflicted with a disease or disorder or the donor is administered an agent which induces mobilization of bone-marrow cells to peripheral blood.

In a further aspect, the subject or donor is administered granulocyte-colony stimulating factor (G-CSF), granulocyte-macrophage colony-stimulating factor (GM-CSF), 1,1′41,4-Phenylenebis(methylene)]-bis[1,4,8,11-tetraazacyclotetradecane] (Plerixafor), a salt thereof, and any combination thereof prior to sample collection.

In some aspects, the target cells are expanded by culturing said target cells in a proliferation medium capable of expanding the target cells. In other aspects, the mitochondria-enriched target cells are expanded by culturing the mitochondria-enriched cells in a culture or proliferation medium capable of expanding mitochondria-enriched cells. As used throughout this application, the term “culture or proliferation medium” is a fluid medium such as cell culture media, cell growth media, buffer which provides sustenance to the cells.

The terms “disease” and “disorder” are meant to refer to any affliction that are not considered normal or that are different from a physiological state. Disease and disorders can affect virtually any organ, tissue, or function in the body. Non limiting examples of diseases and condition include cancer, muscle diseases and disorders, glycogen-storage diseases and disorders, vascular endothelium disorder or diseases, brain disorder or brain disease, placental disorder or placental disease, thymus disorder or thymus disease, autoimmune diseases, renal disease or disorder, pancreas disorder or pancreas disease, prostate disorder or prostate disease, kidney disorder or kidney disease, blood disorder or blood disease, heart disease or heart disorder, skin disorder or skin disease, immune and inflammatory diseases and disorders, bone disease or bone disorder, gastro-intestinal disease or gastro-intestinal disorder, and eye disease or eye disorder. In certain aspects, the disease or disorder is a mitochondrial disease or disorder.

As used herein the term “a subject afflicted with a disease or disorder” or “a subject afflicted having a disease or disorder” refers to a human subject experiencing debilitating effects caused by certain conditions. The disorder may refer to cancer, age related disorders, renal disease, pancreatic diseases, liver diseases, muscle disorders, brain disease or primary mitochondrial diseases, secondary mitochondrial dysfunction, as well as other disease or disorders.

As used herein, the term “ex-vivo method” refers to a method where the steps are performed exclusively outside the human body.

In certain aspects, the target cells, which may be stem cells, are obtained from a subject not afflicted with a disease or disorder or a donor, and the target cells have (i) a normal rate of oxygen (O₂) consumption; (ii) a normal content or activity level of citrate synthase; (iii) a normal rate of adenosine triphosphate (ATP) production; or (iv) any combination of (i), (ii) and (iii).

In certain aspects, the target cells, which may be stem cells, are obtained from a subject afflicted with a disease or disorder or a donor, and the target cells have (i) a decreased rate of oxygen (O₂) consumption; (ii) a decreased content or activity level of citrate synthase; (iii) a decreased rate of adenosine triphosphate (ATP) production; or (iv) any combination of (i), (ii) and (iii), as compared to a subject not afflicted with a disease or disorder.

In certain aspects, the mitochondria-enriched target cells have (i) an increased rate of oxygen (O₂) consumption; (ii) an increased content or activity level of citrate synthase; (iii) an increased rate of adenosine triphosphate (ATP) production; (iv) an increased mitochondrial DNA content (v) a lower level of heteroplasmy or (vi) any combination of (i), (ii), (iii) (iv) and (v) as compared to the target cells.

The term “increased rate of oxygen (O₂) consumption” as used herein refers to a rate of oxygen (O₂) consumption which is detectably higher than the rate of oxygen (O₂) consumption prior to mitochondria enrichment.

The term “increased content or activity level of citrate synthase” as used herein refers to a content or activity level of citrate synthase which is detectably higher than the content value or activity level of citrate synthase prior to mitochondria enrichment.

The term “increased rate of adenosine triphosphate (ATP) production” as used herein refers to a rate of adenosine triphosphate (ATP) production which is detectably higher than the rate of adenosine triphosphate (ATP) production prior to mitochondria enrichment. There are two forms of MAO: MAO-A and MAO-B. MAO-A is an enzyme which

catalyzes the oxidative deamination of amines, such as dopamine, norepinephrine, and serotonin. MAO-A is mainly located in the outer membrane of mitochondria but is also found in the cytosol. MAO-B catalyzes the oxidative deamination of biogenic and xenobiotic amines and plays an important role in the catabolism of neuroactive and vasoactive amines in the central nervous system and peripheral tissues (such as dopamine) and preferentially degrades benzylamine and phenethylamine. MAO-B is located in the outer membrane of mitochondria. Both MAO-A and MAO-B are also located in various tissues with high levels in the placenta.

In an additional embodiment, the present invention provides method of determining enrichment of a cell with placental mitochondria by determining levels of MonoAmine oxidase A (MAO-A), and/or MonoAmine oxidase B (MAO-B), in the cell, wherein cells enriched with placental mitochondria have increased levels of MAO-A and/or MAO-B compared with cells that are not enriched. In one aspect, the cells are stem cells, progenitor cells or bone marrow derived stem cells, pluripotent stem cells, embryonic stem cells, induced pluripotent stem cells, mesenchymal stem cells, hematopoietic stem cells, hematopoietic progenitor cells, myeloid progenitor cells, lymphoid progenitor cells, megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, myelopoietic cells, erythropoietic cells or any combination thereof. In certain aspects, the cells are CD34+ cells. In an additional aspect, the cells were enriched by contacting the cells with mitochondria. In certain aspects, the placental mitochondria are fresh, frozen or freeze-thawed mitochondria. In a further aspect, the MAO-A and/or MAO-B levels are determined by mass spectroscopy.

In a further embodiment, the present invention provides a method for determining enrichment of a cell with exogenous mitochondria by determining levels of glycerol-3-phosphate dehydrogenase wherein cells enriched with mitochondria have increased levels of glycerol-3-phosphate dehydrogenase compared with cells that are not enriched. In one aspect, the cells are stem cells, progenitor cells or bone marrow derived stem cells, pluripotent stem cells, embryonic stem cells, induced pluripotent stem cells, mesenchymal stem cells, hematopoietic stem cells, hematopoietic progenitor cells, myeloid progenitor cells, lymphoid progenitor cells, megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, myelopoietic cells, erythropoietic cells or any combination thereof. In various aspects, the cells are CD34+ cells. In an additional aspect, the cells were enriched by contacting the cells with mitochondria. In certain aspects, the mitochondria are fresh, frozen or freeze-thawed mitochondria. In various aspects, the cells are enriched with placental mitochondria or mitochondria derived from blood. In a further aspect, the cells are enriched with placental mitochondria.

In one embodiment, the present invention provides a kit for identifying cells enriched with mitochondria with a metabolic substrate; and instructions for use. In one aspect, the substrate is tryptamine, D,L-a-glycerol PO₄, succinate, or a combination thereof. In an additional aspect, the mitochondria are placental mitochondria or mitochondria derived from blood.

In one embodiment, the present invention provides a method of determining enrichment of a cell with exogenous mitochondria by determining mitochondrial enrichment after contacting the cell with a metabolic substrate by colorimetric assay, determining levels of MonoAmine oxidase A (MAO-A) and/or MonoAmine oxidase B (MAO-B) in the cell, and/or determining levels of glycerol-3-phosphate dehydrogenase in the cell, wherein cells enriched with mitochondria have increased MonoAmine oxidase A (MAO-A), MonoAmine oxidase B (MAO-B), and/or glycerol-3-phosphate dehydrogenase levels, respectively, as compared with cells that are not enriched with mitochondria wherein the colorimetric assay is measured by absorbance and wherein an increase in absorbance indicates mitochondrial enrichment.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1

### Placental Mitochondira Utilization of Substrates

The ability of isolated placental mitochondria to utilize different substrates was assessed. Mitochondria were isolated from fresh placenta. The isolated mitochondria were incubated with 31 substrates and their ability to utilize the different substrates was assayed by MitoPlate (Biolog). Briefly, the MitoPlate assess mitochondrial function by measuring the rates of electron flow into and through the electron transport chain from metabolic substrates that produce NADH or FADH₂. Each substrate follows a different route, using different transporters to enter the mitochondria and different dehydrogenases to produce NADH or FADH₂. From the NADH or FADH₂ the electrons travel to respiratory complex 1 or 2 and then to the distal portion of the electron transport chain where a tetrazolium redox dye (MC) acts as a terminal electron acceptor that turns purple upon reduction.

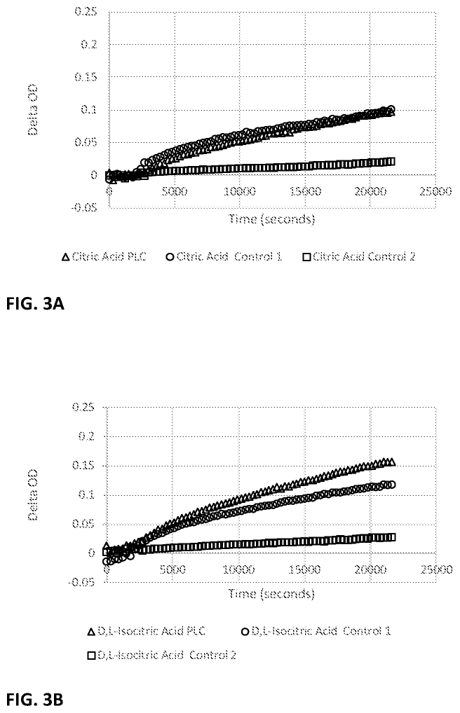

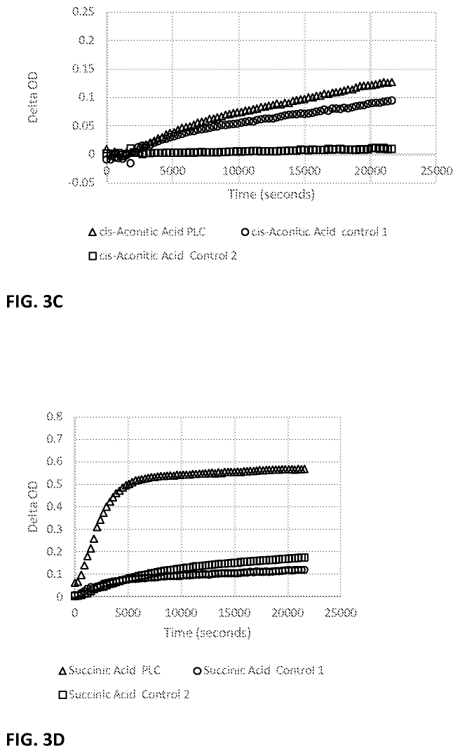

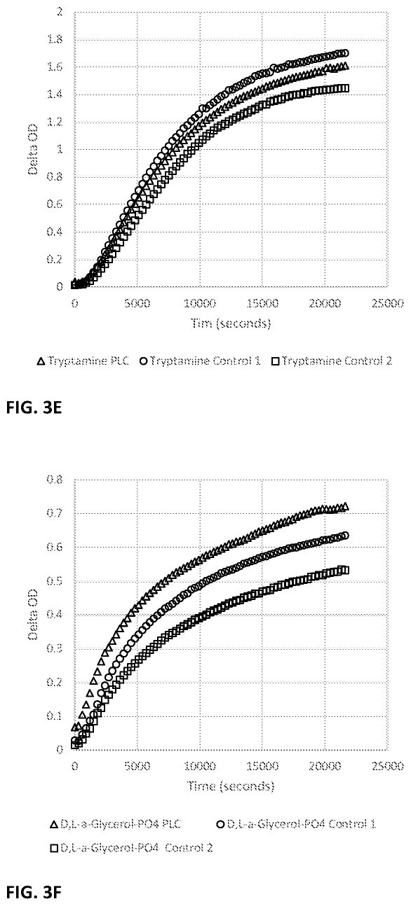

Two controls were used. Control 1 were mitochondria treated with Carbonyl cyanide-4 (trifluoromethoxy) phenylhydrazone (FCCP). FCCP, is an uncoupling agent that collapses the proton gradient and disrupts the mitochondrial membrane potential. Control 2 were isolated mitochondria suspended in water and vortexed thereby having damaged membrane integrity. The results indicate that the isolated functional placental mitochondria were able to utilize Citric acid, D,L-Isocitric acid, Cis-aconitic acid, Succinic acid, Tryptamine, and D,L-a-glycerol PO₄ (FIG. 3).

Additionally, control 1 was able to utilize Citric acid, D,L-Isocitric acid, Cis-aconitic acid, Tryptamine, and D,L-a-glycerol PO₄ but was unable to utilize succinic acid.

### Example 2

### Mitochondria-Enriched Cell Utilization of Substrates

Enriched cells were tested for their ability to utilize the substrates described in Example 1. A healthy subject not afflicted with a mitochondrial disease was administered G-CSF+ Plerixafor to induce mobilization of bone-marrow cells to the peripheral blood (PB). Patient's blood stem cells were collected by apheresis and CD34+ hematopoietic stem cells (HSPCs) were isolated. CD34+ were untreated (NT) or augmented with frozen and thawed healthy mitochondria isolated from the blood or placenta of a healthy donor. Briefly, the cells were mixed with mitochondria from blood or placenta at a dose of 4.4 mU CS activity/million cells (BLD 4.4 and PLC 4.4, respectively), centrifuged at 7000 g and re-suspended. The cells were incubated for 24 hours at room temperature, followed by two washes with PBS. Enrichment was verified by an increase in COX-1 protein (non-treated cells vs. augmented cells).

Mitochondrial function was assayed by measuring the rates of electron flow into and through the electron transport chain from metabolic substrates that produce NADH or FADH₂ using Mitoplate (BIOLOG).

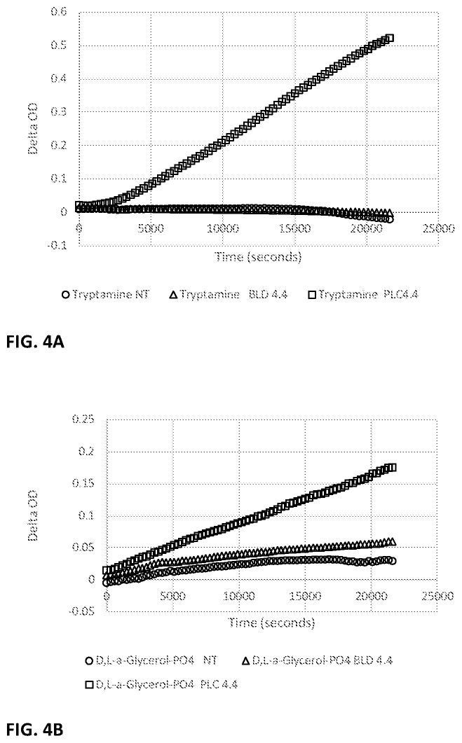

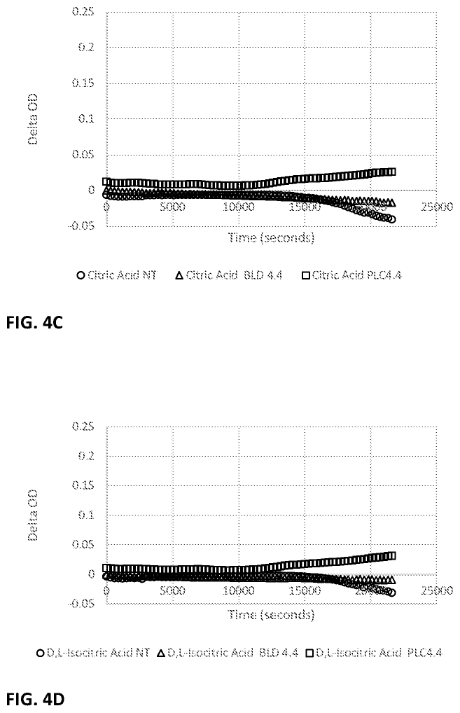

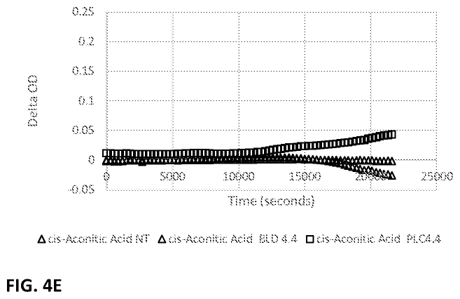

NT cells were used as control. The NT cells and the enriched cells (BLD 4.4 and PLC 4.4) were permeabilized with saponin (Sigma SAE0073), loaded into the MitoPlate and then analyzed for their ability to utilize the substrates (FIG. 4). The results indicated that CD34+ cells enriched with placenta-derived mitochondria (PLC 4.4) were able to utilize tryptamine, D, L-a-glycerol PO₄ (FIG. 4), and succinic acid (data not shown) as substrates. The most significant result was that PLC-enriched cells were able to utilize Tryptamine whereas NT cells and BLD-enriched cells did not.

Additionally, while both blood and placenta mitochondrial enrichment improved the ability of cells to utilize D, L-a-glycerol PO₄ compared to NT cells, BLD-enriched cells did not utilize D, L-a-glycerol PO₄ as efficiently as PLC enriched cells.

Moreover, it was shown in Example 1 that isolated functional placental mitochondria were able to utilize Tryptamine, L-a-glycerol PO₄, citric acid, isocitric acid, cis-aconitic acid, and succinic acid. However, in this experiment, enrichment of cells with mitochondria from placenta or blood, did not show an increase the cells' ability to utilize citric acid, isocitric acid, and cis-aconitic acid.

### Example 3

### Analysis of Mitochondria

Mass spectrometry (MS) was performed to determine the presence of tryptamine utilizing enzymes in mitochondria isolated from the blood and placenta in Example 2. 10 micrograms of each mitochondria sample (3 BLD—mitochondria samples and 3 PLC—mitochondria samples) was extracted in 8M Urea followed by sonication. The extracted proteins were reduced by carbaamidomethylated and digested by trypsin. The resulting peptides were analyzed by LC-MS/MS on Q-Exactive HF (Thermo) and identified by Discoverer 1.4 with the search algorithm: Sequest (Thermo) search engine against the Human uniprot database. All the identified peptides were filtered with high confidence. Semi quantitation was done by calculating the peak area of each peptide based on its extracted ion currents (XICs). For proteins represented by more than 3 peptides the expression intensity of the protein is the average of the three most intense peptides. The protein expression intensities in table 1 are presented in log 2 with the reduction of the background noise. The number of peptides representing each protein is written in brackets.

TABLE 1

| | MAOA | MAOB | ALDH1B1 | ALDH2 |
|---|---|---|---|---|
| BLD-mitochondria (#1) | 0 [0] | 0 [0] | 0 [0] | 25.6 [8] |
| BLD-mitochondria (#2) | 24.4 [2] | 24.7[5] | 0 [0] | 26.5 [13] |
| BLD-mitochondria (#3) | 27.9 [4] | 28.7 [21] | 0 [0] | 24.3 [3] |
| PLC-mitochondria (#1) | 34.1 [45] | 33.2 [16] | 26.9 [7] | 27.5 [10] |
| PLC-mitochondria (#2) | 34.2 [54] | 33.5 [10] | 25.9 [1] | 26.2 [7] |
| PLC-mitochondria (#3) | 32.6 [41] | 31.7 [10] | 24.1 [2] | 25.4 [7] |

The results corroborated that higher levels of MAOA, MAOB and ALDH1B1 were found in the mitochondria isolated from placenta compared to mitochondria isolated from blood.

### Example 4

### Tryptamine Utilization

To test that location of the tryptamine utilizing enzymes, a MitoPlate assay was performed. Peripheral blood mononuclear cells (PBMCs) were enriched with a dose of 4.4 mU CS activity/million cells of mitochondria from placenta (PLC), or enriched with a dose of 4.4 mU CS activity/million cells of mitochondria from placenta that were filtered prior to augmentation (Filtered). The Filtered sample was prepared by passing the sample through a 0.22 um filter to exclude mitochondria (typically ranging in size from 0.5 micrometer to 1 micrometer). Non-treated PBMCs were used as control (NT).

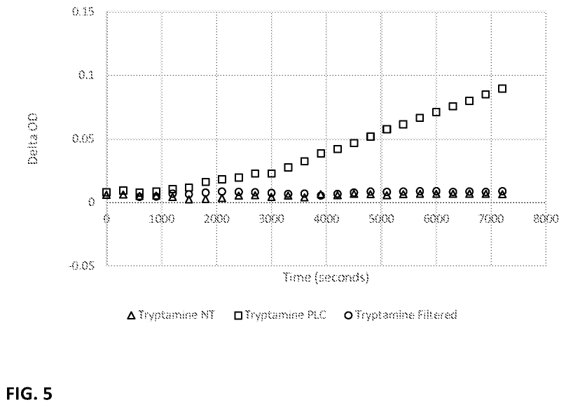

The results shown in FIG. 5 indicate that the tryptamine utilizing enzymes are likely located in mitochondria or bound to the mitochondrial membrane and are not in the fraction that passes through the filter (e.g., cytoplasmic enzymes).

### Example 5

### Fresh and Frozen-Thawed Mitochondria can Utilize Tryptamine

The ability of fresh isolated mitochondria to utilize tryptamine was compared to the ability of frozen-thawed (frozen and thawed, F&T) mitochondria to utilize tryptamine.

Both fresh and F&T mitochondria were isolated from a human placenta. F&T mitochondria were frozen at a temperature of −196° C. for 10 min and thawed at room temperature. The isolated mitochondria were incubated with tryptamine in MitoPlate.

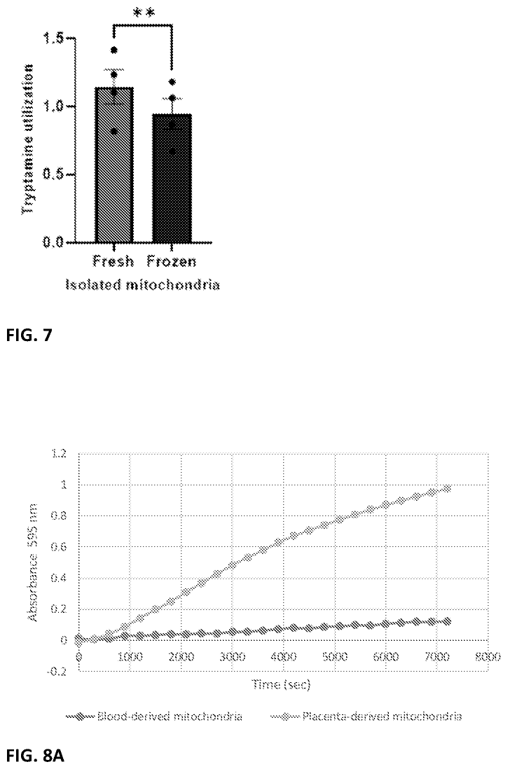

The results shown in FIG. 7 indicate that both fresh and F&T mitochondria utilize Tryptamine.

### Example 6

### Tryptamine Utilization by Mitochondria Enriched Cells

Cell augmentation with placental mitochondria were further verified using a Seahorse XFe96 Analyzer. In this assay oxygen consumption rate (OCR) was measured using specific substrates of complex I (CI) and complex II (CII) in the cell lines KG1a, LCL (GM18456 from Coreille, Pearson patient) and CD34+(Hemacare lot: 18049698). 10×10⁶ cells of LCL, KG1a, or CD34+ were augmented with placental mitochondria at a dose of 0.88 mU or 4.4 mU CS activity/million cells. Augmented cells were compared to non-augmented cells. Cells were treated with permeabilization agent (Saponin) and were maintained in a RPMI medium or MAS buffer (70 mM sucrose, 220 mM mannitol, 5 mM KH2PO4, 5 mM MgCl2, 1 mM EGTA, 2 mM HEPES pH 7.4), supplemented with Glucose (10 mM), Pyruvate (1 mM) and Glutamate (2 mM). Pre-coated Seahorse XF96 microplate was loaded with 50 μl medium containing 300k cells per well of KG1a, 300k cells per well of CD34+, or 150k cells per well of LCL. The plate was centrifuged at 200 g for 1 min. and an additional 130 μl of RPMI medium or MAS+ substrate was added to each well. Tryptamine was used as CI specific substrate and Succinate as CII specific substrate. For the LCL cells and the KG1a cells, ADP was injected at port A (5 mM final concentration), Tryptamine at port B (60 mM), and Succinate at port C (10 mmM) For the CD34+ cells, ADP (5 mM final concentration) was injected at port A with Tryptamine (60 mM). Mix and measure times were 3 min. and 3 min, respectively. Wave software (Agilent) was used to analyze OCR rates. OCR measurements were normalized to the background (medium and substrates without cells).

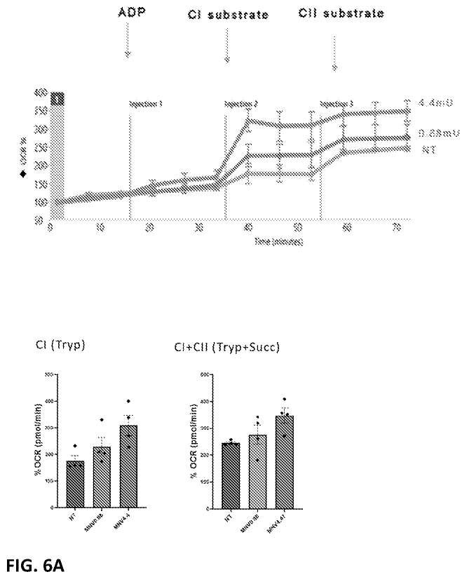

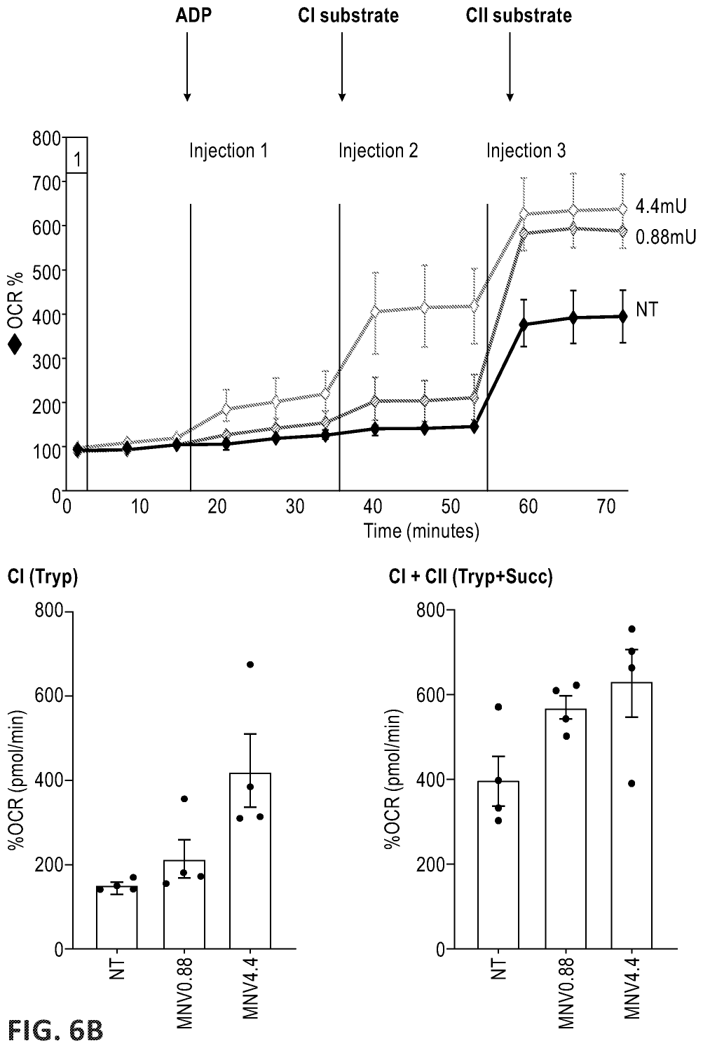

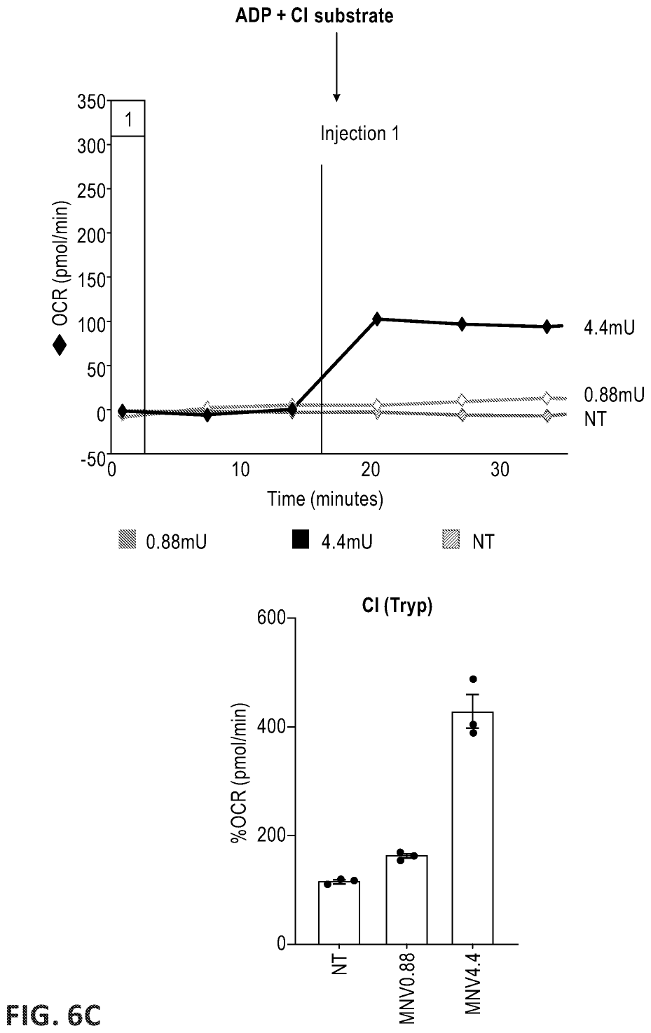

The results show that upon addition of tryptamine as CI substrate, KG1a cells augmented with mitochondria dose of 0.88 mU or 4.4 mU CS activity/million cells had an increase in OCR levels of 29% and 74% respectively, compared to the NT control cells. Moreover, upon addition of both tryptamine and succinate, CI and CII combined respiration rate (OCR) were also increased in the 0.88 mU and 4.4 mU augmented cells by 12% and 41% respectively, compared to control non augmented cells (NT) (FIG. 6A).

Upon addition of tryptamine as CI substrate, LCL cells augmented with mitochondria dose of 0.88 mU or 4.4 mU CS activity/million cells had an increase in OCR levels of 43% and 182% respectively, compared to the NT control cells. Upon addition of both tryptamine and succinate, CI and CII combined OCR were also increased in the 0.88 mU and 4.4 mU augmented cells by 43% and 57% respectively, compared to control non augmented cells (NT) (FIG. 6B).

For CD34+ cells, the results show that upon addition of tryptamine, CD34+ cells augmented with mitochondrial doses of 0.88 mU and 4.4 mU CS activity/million cells had an increase in OCR levels of 39% and 271%, respectively (FIG. 6C).

### Example 7

### Detection of Enzyme Expression to Assess Mitochondrial Enrichment

To detect placental mitochondrial enrichment in cells the presence of MAO enzymes is determined. Briefly, augmented and non-augmented cells will be homogenized using MAO Assay buffer. The homogenate is centrifuged (10,000×g for 4 minutes at 4C) and the supernatant is collected. Controls, standards (H2O2), reaction buffer (assay buffer, MAO substrate, developer and probe) and background reaction mix (assay buffer, developer and probe) are prepared. Sample supernatant (40 μl) is added to the wells of a reaction plate. To measure total MAO activity, 10p1 of assay buffer is added to designated wells. To measure MAO-A activity, 10 μl of 10 μM Selegiline is added to designated wells. To measure MAO-B activity, 10 μL of 10 μM Clorgyline is added to designated wells. Reaction mix (50 μl) is added to each well and background reaction mix (50 μl) is added to the background control sample wells. Fluorescence (Ex/Em=535/587 nm) is measured in kinetic mode at 25° C. for 60 minutes. An increase in total MAO, MAO-A and/or MAO-B activity in augmented cells is indicative of cell mitochondrial enrichment.

### Example 8

### Maoa Staining Technique for Detection of Plc-Mitochondria

Since MAO-A is a mitochondrial protein that is detected in placenta cells and not in CD34+ cells, a fluorescent labeling method was developed specific for the detection of placenta mitochondria (immunofluorescence-based assay) over the background of hematopoietic stem cells. This method enabled an imaging-based analysis to quantitively measure augmentation in a single cell resolution.

MAO-A antibody (Abcam cat. #ab200752) and TOMM20 antibody (Abcam cat. #ab210047) were used. TOMM20 is a universal mitochondria antibody, which was used as an internal positive control of the assay. TOMM20 was found to completely colocalize with MitoTracker (Invitrogen, Catalog #M22426) which is a red-fluorescent dye that stains mitochondria in live cells.

Healthy CD34+ cells were incubated with isolated placental mitochondria at a mitochondria to cell ratio of 4.4 mU (by CS activity) per million cells. After 24 hr incubation, CD34+ cells were washed three times. NT cells were used as a control (e.g., cells without addition of mitochondria). Cells were immunostained for 30 min on ice (gentle shaking), with the antibodies in 1% BSA, TOMM20-AF405 (final dilution of 1:5000) and Ab MAOA (for final dilution of 1:50).

The cells were imaged with Amnis IMAGESTREAM X MARKII. The data was analyzed with IDEAS® Analysis Software.

Immunofluorescence with MAO-A was highly sensitive and highly specific, therefore the cells enriched with placental mitochondria were stained whereas the non-treated cells were not stained.

### Example 9

### Succinate Utilization

Mitochondria were isolated from human placenta and human peripheral blood mononuclear cells (PBMCs). The ability of the mitochondria to utilize the succinate substrate was assayed by MitoPlate (Biolog).

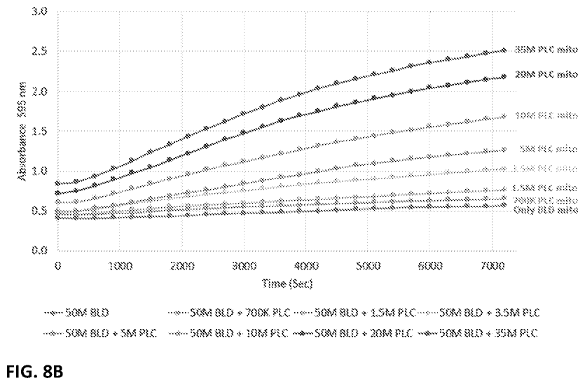

The results shown in FIG. 8A indicate that mitochondria isolated from placenta have a higher succinate-utilization activity compared to mitochondria isolated from blood.

Since both blood-derived mitochondria and placenta-derived mitochondria have the ability to utilize succinate (FIG. 8A), the sensitivity level of the method to different ratios of exogenous mitochondria in augmented cells was tested.

The ability of placenta-derived mitochondria to utilize succinate was tested on the background of mitochondria isolated from PBMCs. The background control used was of mitochondria isolated from PBMCs. 50M blood-derived mitochondria particles were used as an equivalent of mitochondria isolated from 1M CD34 cells. Rising amounts of placenta-derived mitochondria particles (750k to 35M) were added to the background of 50M blood-derived mitochondria particles to assess total succinate utilization activity.

As can be seen in FIG. 8B, placenta-derived mitochondria ability to utilize succinate over the background activity of the 50M blood-derived mitochondria particles was evident at high and low concentrations of placenta-derived mitochondria.

Although the invention has been described with reference to the above examples, it will be understood that modifications and variations are encompassed within the spirit and scope of the invention. Accordingly, the invention is limited only by the following claims.

1. A method of determining enrichment of a cell with exogenous mitochondria comprising:
a) contacting the cell with a metabolic substrate; and
b) determining electron transfer in the cell following contacting with the metabolic substrate.

2. The method of claim 1, wherein determining the electron transfer is by colorimetric assay, fluorescent assay, luminescent assay, or oxygen consumption.

3. The method of claim 1, wherein the cells are enriched with placental mitochondria or mitochondria derived from blood.

4. (canceled)

5. The method of claim 1, wherein the cells are selected from the group consisting of stem cells, progenitor cells or bone marrow derived stem cells, pluripotent stem cells, embryonic stem cells, induced pluripotent stem cells, mesenchymal stem cells, hematopoietic stem cells, hematopoietic progenitor cells, myeloid progenitor cells, lymphoid progenitor cells, megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, myelopoietic cells, erythropoietic cells and any combination thereof.

6. The method of claim 5, wherein the cells are CD34+ cells.

7. The method of claim 1, wherein the metabolic substrate is selected from the group consisting of tryptamine, D,L-a-glycerol PO₄, succinate and a combination thereof.

8-9. (canceled)

10. The method of claim 1, wherein the cells were enriched by contacting the cells with exogenous mitochondria.

11. The method of claim 1, wherein the colorimetric assay is measured by absorbance.

12. The method of claim 11, wherein increased absorbance indicates the cell is enriched.

13. The method of claim 1, wherein contacting the cells with the metabolic substrate produces NADH and/or FADH₂.

14. The method of claim 1, wherein the mitochondria are fresh, frozen-thawed, or any combination thereof.

15. (canceled)

16. The method of claim 29, wherein the cells are selected from the group consisting of stem cells, progenitor cells or bone marrow derived stem cells, pluripotent stem cells, embryonic stem cells, induced pluripotent stem cells, mesenchymal stem cells, hematopoietic stem cells, hematopoietic progenitor cells, myeloid progenitor cells, lymphoid progenitor cells, megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, myelopoietic cells, erythropoietic cells and any combination thereof.

17. The method of claim 16, wherein the cells are CD34+ cells.

18. The method of claim 29, the cells were enriched by contacting the cells with mitochondria.

19. The method of claim 29, wherein the MAO-A and/or MAO-B levels are determined by mass spectroscopy.

20-23. (canceled)

24. The method of claim 29, wherein the cells are enriched with placental mitochondria or mitochondria derived from blood.

25. The method of claim 24, wherein the cells are enriched with placental mitochondria.

26-28. (canceled)

29. A method of determining enrichment of a cell with exogenous mitochondria comprising
determining electron transfer after contacting the cell with a metabolic substrate, determining levels of MonoAmine oxidase A (MAO-A) and/or MonoAmine oxidase B (MAO-B) in the cell, and/or determining levels of glycerol-3-phosphate dehydrogenase in the cell, and
wherein after contacting the cell with a metabolic substrate, cells enriched with mitochondria have increased levels of electron transfer, MonoAmine oxidase A (MAO-A), MonoAmine oxidase B (MAO-B), and/or glycerol-3-phosphate dehydrogenase, as compared with cells that are not enriched with mitochondria.

30. The method of claim 29, wherein determining the electron transfer is by colorimetric assay.

31. The method of claim 30, wherein the colorimetric assay is measured by absorbance and wherein an increase in absorbance indicates mitochondrial enrichment.
